(19) United States
(12) Patent Application Publication
(10) Pub. No.: US 2004/0194157 A1
(43) Pub. Date: Sep. 30, 2004

(54) Animal with surgically modified gastrointestinal tract and method for study of weight reduction

(76) Inventor: Michael M. Meguid, East Syracuse, NY (US)

Correspondence Address: Sander Rabin, Sander Rabin MD JD & Associates, 2 Irving Place, Troy, NY, 12180-4417, US

(21) Appl. No.: 10/802,996

(22) Filed: Mar. 17, 2004

Related U.S. Application Data
(60) Provisional application No. 60/457,213, filed on Mar. 24, 2003.

(51) Int. Cl. A01K 67/00; A01K 67/033; A61F 5/24

(52) U.S. Cl. 800/8; 128/96.1

(57) ABSTRACT

The invention comprises an animal having a presurgical substantially normal gastrointestinal tract, which gastrointestinal tract has been surgically modified such that postsurgically there is a reduction of the volume of the stomach of the gastrointestinal tract, a reduction in the digestive area of the gastrointestinal tract, a reduction in the co-mingling of food with gastric, biliary and pancreatic juices, a reduction in the presurgical gastric output of the peptide ghrelin, a reduction in the threshold for satiety, a permanent reduction in presurgical weight, and an induction of a condition of malabsorption. The surgically-altered animal may be adapted for use as an animal model in a method wherein the biological mechanisms underlying obesity and its reduction may be investigated; and, in which the molecular biological effects of surgical intervention for obesity may be investigated; and, in which the efficacy of noninvasive alternatives to surgical intervention for obesity may be investigated.

### BACKGROUND OF THE INVENTION

1. Technical Field

The present invention relates generally to an animal model created by a surgical modification of an animal's gastrointestinal tract, and the use of the animal model in a method for studying the biological mechanisms of obesity and the reduction of obesity.

2. Related Art

Obesity is a life-threatening public health dilemma whose incidence and prevalence has increased at an alarming rate in developed countries. In the United States, obesity affects about 97 million American adults, corresponding to about 55 percent of the population. These individuals are at increased risk of obesity-associated diseases, such as, for example, hypertension, lipid disorders, diabetes, coronary heart disease, stroke, gallbladder disease, osteoarthritis, sleep apnea, respiratory problems, and certain cancers. The total costs attributable to obesity-related disease has been estimated as approaching $100 billion annually.

The first Federal guidelines on the identification, evaluation, and treatment of overweight obese in adults were released in 1998 by the National Heart, Lung, and Blood Institute, in cooperation with the National Institute of Diabetes and Digestive and Kidney Diseases. According to the guidelines, the clinical assessment of obesity involves evaluation of three key measures—body mass index (“BMI”), waist circumference, and a person's risk factors for diseases and conditions associated with obesity.

The BMI is an anthropometric index, calculated as the ratio of a person's weight to the square of a person's height (lb/in² or kg/m²). The BMI is strongly correlated with total body fat content in male and female adults, and has become the medical standard for assessing the clinical propriety of a person's weight. Obesity is present in persons having a BMI of 30 and above. Clinically severe obesity, formerly called morbid obesity, is present in persons having a BMI in excess of 40.

Surgical intervention is indicated in persons with a BMI in excess of 35 when noninvasive methods have failed and the person either has, or is at high risk for, life-threatening obesity-associated diseases. The degree of weight loss achieved postoperatively and its permanence are hallmarks of successful surgical intervention. However, the biological mechanisms responsible for achieving a significant and permanent reduction in the weight of persons who have undergone surgical intervention for clinically severe obesity, or obesity in the setting of obesity-associated diseases, are incompletely known.

Accordingly, there exists a need for an animal model and a reproducible, standardized method employing the animal model in which:

the biological mechanisms underlying obesity, obesity in the setting of obesity-associated diseases, and clinically severe obesity can be investigated; and,

the molecular biological effects of surgical intervention for clinically severe obesity, and obesity in the setting of obesity-associated diseases can be investigated; and,

the efficacy of noninvasive alternatives to surgical intervention for obesity, obesity in the setting of obesity-associated diseases, and clinically severe obesity can be investigated.

### SUMMARY OF THE INVENTION

The present invention comprises an animal model for the study of obesity comprising a surgically modified animal comprising an animal having a preoperative weight, a preoperative state of endogenous ghrelin output and a preoperative substantially normal animal gastrointestinal system that has been surgically modified, wherein said surgical modification reduces the volume of the stomach of said gastrointestinal tract and reduces the digestive area of said gastrointestinal tract; and, wherein postoperatively, said surgically modified animal exhibits a substantially permanent weight reduction relative to its preoperative weight and a substantially permanent reduction in said preoperative state of endogenous ghrelin output. The present invention also includes a method that uses the animal model for investigating the biological mechanisms of obesity and obesity reduction.

### BRIEF DESCRIPTION OF THE DRAWINGS

FIG. 1 is a schematic illustration of a portion of a substantially normal animal gastrointestinal tract beginning approximately at the terminal esophagus and ending approximately at the mid-jejunum.

FIG. 2 is a schematic illustration of a portion of an animal gastrointestinal tract, schematically illustrating the locations of a surgical division of the jejunum, a surgical line of closure of the gastric fundus, a gastrojejunostomy, and a jejunojejunostomy in a Roux-en-Y gastroplasty

FIG. 3 is a schematic illustration of a portion of an animal gastrointestinal tract that has been reconstructed using a Roux-en-Y gastroplasty.

FIG. 4 contains a graph of the effect of gastric bypass or a sham operation on the body weight of Zucker rats as a function of time in days.

FIG. 5 is a schematic illustration of the end-result of a divisional Roux-en-Y gastroplasty.

FIG. 6 is a schematic illustration of the end result of a vertically banded gastroplasty.

FIG. 7 is a schematic illustration of the end result of a gastric banding procedure.

### DETAILED DESCRIPTION OF THE INVENTION

As used herein, the terms “surgical modification of the gastrointestinal tract” and “surgically modified gastrointestinal tract” include, but are not limited, to the following surgical procedures: bariatric surgeries, gastric banding, lap-band adjustable gastric banding, gastric reduction, gastric by-pass, gastrectomy, gastroplasty, Roux-en-Y gastroplasty, vertical banded gastroplasty, silastic ringed vertical gastroplasty, intestinal bypass, restriction operations, and weight-loss surgery.

As used herein, the term “biological mechanisms,” includes, but is not limited to neurobiological mechanisms, physiological mechanisms, pathophysiological mechanisms, molecular biological mechanisms, biochemical mechanisms, metabolic mechanisms and genetic mechanisms.

As used herein, the term “biological factors” includes, but is not limited to, measurements or assays of serum, tissue, or body fluid concentrations or densities of, inter alia: glucose; glucagon; free fatty acids; triglycerides; cholesterols; high-density lipoproteins; low-density lipoproteins; insulin; steroids; sterols; ghrelin; neurohormonal or neuromodulatory peptides, such as, for example, leptin, neuropeptide Y, or neuropeptide YY; monoamine neurotransmitters, such as, for example, dopamine and serotonins; 11_hydroxysteroid dehydrogenase type 1; 5-hydioxytryptamine-1 B R; angiotensin-converting enzyme; Agouti-related peptides; cholecystokinins; C-reactive proteins, corticotropin-releasing hormone; gamma amino butyric acid; growth hormone; growth hormone secretagogues; interleukins; melanocyte stimulating hormones; melanocortin hormone; thyroid hormones; epinephrine; norepinephrine; tissue-plasminogen activators; pro-opiomelanocortin; interleukins; tumor necrosis factors; adiponectin; resistin; hydroximethylglutaryl Co-A; estrogen; testosterone; prolactin; and, vasopressin.

As used herein, the term “biological factors” further includes, but is not limited to, analyses, including microarray analyses, measurements, and assays of: genetic expression profiles for the synthesis of the foregoing substances and classes of substances; messenger RNA expression coding for the synthesis of the foregoing substances and classes of substances; cocaine and amphetamine related transcripts; cell surface receptors for the foregoing substances and classes of substances, including, for example, β-adrenergic receptors, orexigenic, and, anorectic receptors; body weight; body mass index; body tissue weights, including, for example, the weights of retro-peritoneal fat pads, epididymal fat pads; tissue fat concentrations, including, for example, liver fat concentration; blood pressure; and, immunohistochemical staining of tissues, such as, for example, hypothalamic and other nervous system tissues, mesenteric fat, retroperitoneal fat and subcutaneous fat.

The beneficial effect of diet in effecting weight loss in obese patients is lost when compliance with a dietary regimen ceases. Similarly, the beneficial effect of pharmacologic agents, such as, for example, appetite suppressants, in effecting weight loss in obese patients is also lost when either compliance with a dosing regimen ceases, or when the pharmacologic agent is no longer taken, or when drug tolerance evolves. As a rule, when there is noncompliance with, or termination of, a dietary or pharmacologic intervention in obesity, lost weight is regained with an accompanying increase in obesity-related morbidity and mortality from obesity-associated diseases, such as, hypertension, dyslipidemias, diabetes, cardiovascular disease, including coronary artery disease, congestive heart failure, renal insufficiency, transient ischemic attacks, stroke, gallbladder disease, osteoarthritis, sleep apnea, respiratory problems, and certain cancers.

Obesity produces changes in the genetic expression profiles of neurohormones acting upon the hypothalamus, such as, for example, ghrelin and leptin, and neurotransmitters, such as, for example, serotonin and dopamine. It is theorized that ghrelin, an endogenous appetite stimulant acting upon the hypothalamus, undergoes a compensatory increase in production by the stomach as a homeostatic response to the reduction in food intake accompanying any diet. The resultant stimulation of the appetite tends to counteract the beneficial effect of the reduction in food intake, and tends to move a person's weight back to the weight disturbed by the diet.

By contrast, the beneficial effect of surgical modification of the gastrointestinal tract, when indicated and performed for obesity in humans, tends to endure, with a permanent loss of weight eventuating in a stable and reduced weight two to three years postoperatively. It has been shown that surgical modification of the gastrointestinal tract is not accompanied by the compensatory increase in the endogenous production of ghrelin seen in with dietary intervention for obesity. Rather, after surgical modification of the gastrointestinal tract, serum ghrelin concentrations are significantly reduced. After surgical modification of the gastrointestinal tract some obesity-induced changes in the genetic expression profiles of neurohormones and other biological factors acting upon the hypothalamus and associated changes in their transcriptional and metabolic products revert to normal. The success of surgical modification of the gastrointestinal tract, when indicated and performed for obesity and its associated diseases in humans, and the ease with which, in particular, laparoscopic gastric by-pass with the Roux-en-Y operation can be performed, have made this procedure one of the most common operations performed in the United States.

The principles of surgical modification of the gastrointestinal tract performed for obesity and its associated diseases are twofold:

[i] a reduction of the volume of the stomach in which food is lodged while undergoing digestion in the stomach, by the establishment of a small gastric pouch to induce early satiety and to minimize the amount of food that can be comfortably ingested; and,

[ii] induction of a degree of malabsorption, by an effective reduction in the digestive area for nutrient absorption and a reduction in the co-mingling of food with gastric, biliary and pancreatic juices.

These two surgical principles do not, however, reveal the detailed biological mechanisms whereby post-operative weight loss occurs, and, more importantly, is sustained.

An understanding of the biological mechanisms involved in the state of permanent weight loss created by surgical modification of the gastrointestinal tract would provide insight into potential pharmacological methods of weight control for those patients who are not candidates for such surgery. Consequently, there is a need for an animal model for investigating the biological mechanisms of obesity and its reduction in the post-surgical state, including, inter alia, the role of the gastric peptide ghrelin, its influence on the hypothalamus, and its interaction with monoamine neurotransmitters such as dopamine and serotonin, and peptides, such as leptin, which has also been identified to influence food intake, body weight, and appetite after surgery.

In accordance with the present invention, the inventor has found that animals having previously substantially normal gastrointestinal systems that undergo a surgical modification of their gastrointestinal tracts, experience a permanent loss of weight, the degree and duration of which correlate with the degree and duration of the weight loss experienced by humans who undergo a similar surgical modification of their gastrointestinal tracts. Animals that have undergone surgical modification of the gastrointestinal tract in accordance with the invention attain a desirable state of homeostasis with respect to their weight that is etiologically similar to that achieved in obese human beings who have undergone analogous surgery to their gastrointestinal tracts.

To study the biological mechanisms of obesity and weight reduction in human obesity, an animal model comprising a surgical modification of the animal's gastrointestinal tract was developed by the present inventor. The model uses an animal having a pre-surgical weight, a presurgical output of ghrelin and other biological factors for obesity, and a pre-surgical substantially normal gastrointestinal tract that is surgically modified, such that post-surgically there is:

[i] a reduction of the volume of the stomach of the animal's gastrointestinal tract;

[ii] a reduction in the digestive area of the animal's gastrointestinal tract;

[iii] a reduction in the co-mingling of food with gastric, biliary and pancreatic juices;

[iv] a permanent reduction in the pre-surgical homeostatic output of ghrelin; and,

[v] a permanent reduction in the animal's presurgical weight.

Alternatively stated, the animal emerges from the surgical modification of its gastrointestinal tract in a homeostatic state of permanent weight reduction relative to its presurgical weight.

The animal model described herein can be very advantageously used, for example, for:

[i] testing hypotheses for the causes of obesity and its treatment, such as, for example, whether peptidergic hypothalamic systems are subject to regulatory influences from the autonomic nervous system and are regulated by monoaminergic neurotransmitters, such as, for example, dopamine and serotonin, to produce a metabolic state conducive to obesity, as occurs with alterations in the ghrelin and leptin signaling pathways; and,

[ii] investigating the biological mechanisms underlying obesity; and,

[iii] investigating the biochemical, genetic and molecular biological effects of surgical modification of the gastrointestinal tract for clinically severe obesity, and obesity in the setting of obesity-associated diseases; and,

[iv] investigating the efficacy of treatments for obesity and noninvasive alternatives to surgical modification of the gastrointestinal tract for clinically severe obesity, and obesity in the setting of obesity-associated diseases; and,

[v] investigating the defunctionalized stomach that eventuates from the surgical modification of the gastrointestinal tract for obesity; and, investigating the molecular biology of ras oncogenes and their relationship to the development of gastric cancer; and, investigating postoperative ulcers, postoperative hemorrhage, and postoperative hydrogen ion. secretion.

Animals suitable for surgical modification of their gastrointestinal tracts, as described or illustrated hereinbelow, include, for example, murine, ovine, porcine, caprine, canine, feline, and primate animals. Such murine, ovine, porcine, caprine, canine, feline, and primate animals may be transgenic, cloned, or genetically engineered to endow them with certain phenotypes; or, they may be naturally occurring or bred for laboratory use.

Surgical modification of the gastrointestinal tract of the animal that is the subject of this invention may be selected from the group comprising bariatric surgeries, gastric banding, lap-band adjustable gastric banding, gastric reduction, gastric by-pass, gastrectomy, gastroplasty, Roux-en-Y gastroplasty, vertical banded gastroplasty, silastic ringed vertical gastroplasty, intestinal bypass, restriction operations, and weight-loss surgery.

The Zucker rat was selected for surgical modification of its gastrointestinal tract to create an exemplary animal model of obesity because its biochemistry in relationship to obesity is well defined. The origin of obesity in the Zucker rat is a missense mutation of the gene coding for leptin receptor. The altered leptin signaling pathway in the Zucker rat diminishes the leptin signaling to the brain, leading to numerous adaptive changes downstream of leptin target cells of the central regulatory systems. Consequently, the Zucker phenotype is expressed as the so called “Zucker syndrome,” among whose features are hyperphagia, large meal sizes, fewer meal numbers, positive energy balance, obesity, and diseases associated with obesity, including, inter alia, diabetes, insulin resistance, hypertension, cardiovascular disease and renal insufficiency and failure. Zucker rats eat up to 36 grams of standardized laboratory rat chow per day, with each meal size being about 3 to 4 grams.

Zucker rats do not show a complete absence of leptin action in intracellular signal transduction, but instead show a reduction in signal transduction associated with leptin. Thus, the altered leptin signaling pathway in the Zucker rat, owing to a single genetic mutation, results in leptin resistance. This resistance is also observed in human obesity, in which it is considered polygenic. The chronic adaptation to the altered leptin signaling pathway in Zucker rats creates the foregoing “Zucker syndrome,” or in the human, the analogous “Syndrome X,” which has all of characteristics associated with human obesity, including, inter alia, diabetes, insulin resistance, hypertension, cardiovascular disease and renal insufficiency and failure. In either the Zucker syndrome or human Syndrome X, the downstream neuronal pathways activated or inhibited by leptin and involved in the regulation of food intake and energy balance represent an important biological mechanism in the pathogenesis of obesity.

As an exemplary animal model for obesity, Zucker rats in a post-surgical state of permanent weight reduction relative to their preoperative weight were created by performing a Roux-en-Y gastroplasty upon their previously substantially normal gastrointestinal tracts. In the description of a non-limiting, exemplary specific method for the study of the biologic mechanisms of obesity to be described shortly, Zucker rats whose gastrointestinal tracts underwent Roux-en-Y gastroplasty were assigned to a group denominated the “gastric bypass” (“GB”) group.

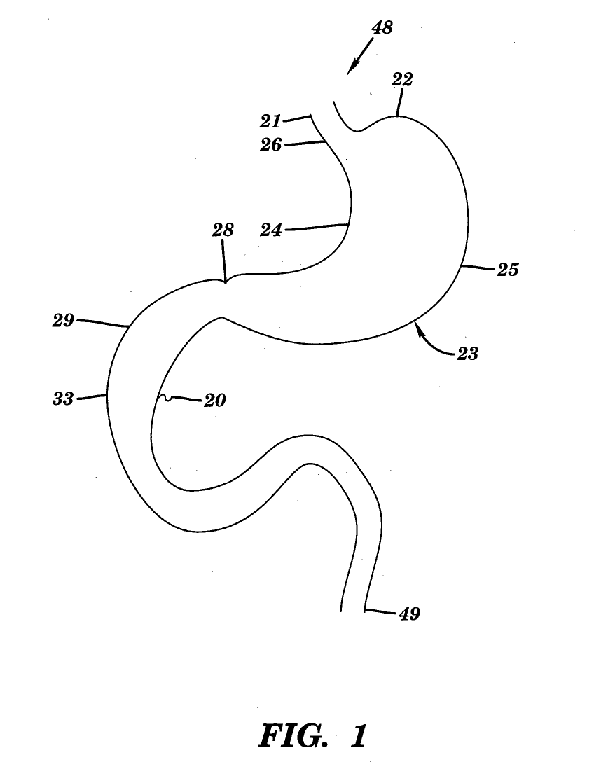

Referring now to the drawings in which like parts are designated by like numerals in the various views, there is shown in FIG. 1 a schematic illustration of a portion of a substantially normal gastrointestinal tract 48 of an animal, beginning at the terminal esophagus 21 and extending to the mid-jejunum 49. Normal gastrointestinal tract 48 is characterized by several anatomical landmarks and regions. Gastroesophageal junction 26 admits food into the stomach 23, having an apical portion 22, called the fundus, and having a contour with a lesser curvature 24 and a greater curvature 25. Partially digested food passes from the pylorus 28 of the stomach into the duodenum 29, the first division of the small intestine, about 25 cm in length, and thence into the jejunum 33, shown as extending to its approximate midpoint 49.

To create an exemplary animal model or GB Zucker rat, a Zucker rat having a substantially normal gastrointestinal tract 48, as shown in FIG. 1, underwent a Roux-en-Y gastroplasty as follows:

a. Anesthesia is administered by intramuscular injection, using a mixture of ketamine and xylazine, in the ratio of 200 mg ketamine to 5 mg xylazine, at a dose of 0.1 ml per 100 g of animal weight;

b. The abdomen is shaved and sterilized in the manner of pre-surgical preparation well known in the surgical arts;

c. The abdomen is opened with a midline epigastric incision of about 4 cm;

d. The terminal esophagus 21, lesser curvature of the stomach 24 and greater curvature of the stomach 25 shown in FIG. 1 are identified;

e. The terminal esophagus 21, lesser curvature 24 of the stomach 23 and greater curvature 25 of the stomach 23 shown in FIG. 1 are dissected free of their surrounding supportive and membranous tissues;

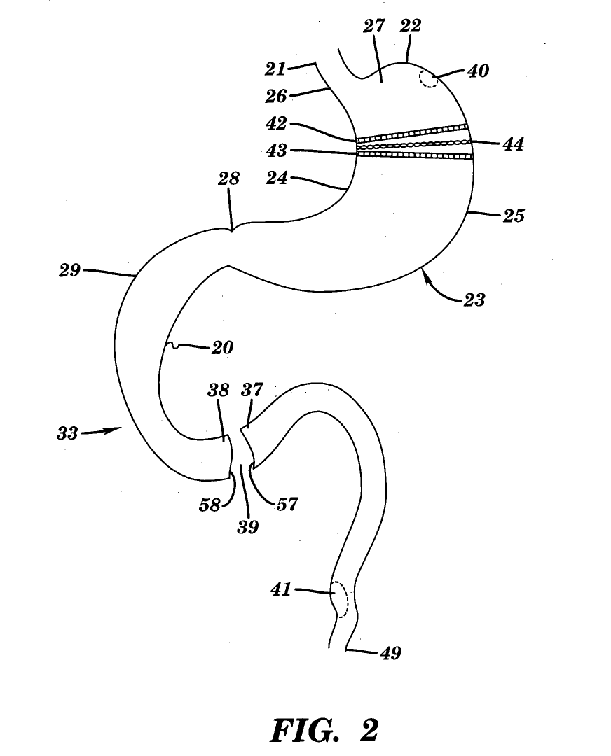

f. As shown schematically in FIG. 2, the gastric fundus 22 of the stomach is closed without transecting the stomach 23, by placing a first row of surgical staples 42 (TRH30-4.8 titanium staples, Ethicon, Cincinnati, Ohio) across the stomach about 2 to 3 mm below the gastroesophageal junction 26, and placing a second row of surgical staples 43 (TRH30-4.8 titanium staples, Ethicon, Cincinnati, Ohio) across the stomach about 4 to 5 mm, below the gastroesophageal junction 26, the first 42 and second 43 rows of surgical staples being reinforced with multiple sutures 44 (4-O polyglactin, Ethicon, Cincinnati, Ohio), thereby creating Roux-en-Y stomach pouch 27 having a volume of about 20% of the volume of the pre-surgical stomach 23;

g. As shown schematically in FIG. 2, the jejunum 33 is divided at a location 39 about 16 cm below the ligament of Treitz 20, into a distal portion 37, having a distal cut end 57, and a proximal portion 38, having a proximal cut end 58;

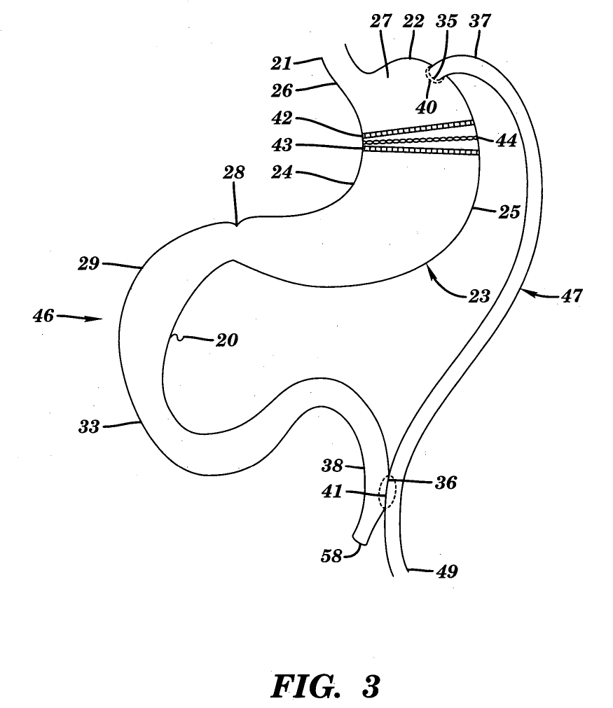

h. As shown schematically in FIG. 3, mobilizing the distal portion 37 (FIG. 2) of the divided jejunum, an end-to-side gastrojejunostomy of about 4 to 5 mm 35, is sewn by hand, using interrupted 5-O polyglactin sutures (Ethicon, Cincinnati, Ohio), thereby joining the distal cut end 57 (FIG. 2) of the distal portion 37 (FIG. 2) of the divided jejunum to the anterior surface of the gastric fundus at site 40 on the anterior surface of the gastric fundus;

i. As shown schematically in FIG. 3, mobilizing the proximal portion of the divided jejunum 38, a side-to-side jejunojejunostomy 36 of about a 7 to 8 mm is sewn by hand at location 41, at a distance of about 10 cm below the site of the gastrojejunostomy 40, thereby joining proximal portion of the divided jejunum to location 41;

j. As shown schematically in FIG. 3, the cut end 58 of the proximal portion of the divided jejunum 38 is closed with running sutures to form a stump.

As shown in FIG. 3, the foregoing surgical steps have the effect of creating a gastrointestinal modification comprising an afferent jejunal limb 46 of the Roux-en-Y gastroplasty measuring about 16 cm from the ligament of Treitz 20—thereby eliminating 80% of the volume of the stomach and 10 cm of the jejunum from participation in digestion—and a Roux-en-Y jejunal limb 47 of the Roux-en-Y gastroplasty, measuring about 10 cm in length from the gastrojejunostomy site 40 to the jejunojejunostomy site 41.

Following the foregoing surgical modification, the midline epigastric incision of the abdomen is closed.

As indicated hereinabove, the Roux-en-Y gastroplasty is an example of a surgical modification of the gastrointestinal tract that may be used:

[i] to induce early satiety by effecting a reduction of the volume of the stomach in which food is lodged while undergoing digestion in the stomach; and,

[ii] to induce a degree of malabsorption by an effecting a reduction in the digestive area of the animal's gastrointestinal tract; and,

[iii] to effect a permanent reduction in the animal's gastric output of ghrelin; and,

[iv] to effect a permanent reduction in the animal's weight relative to its pre-surgical weight.

Other surgical modifications of the gastrointestinal tract of the animal that is the subject of this invention may be selected from the group comprising bariatric surgeries, gastric banding, lap-band adjustable gastric banding, gastric reduction, gastric by-pass, gastrectomy, gastroplasty, Roux-en-Y gastroplasty, vertical banded gastroplasty, silastic ringed vertical gastroplasty, intestinal bypass, restriction operations, and weight-loss surgery.

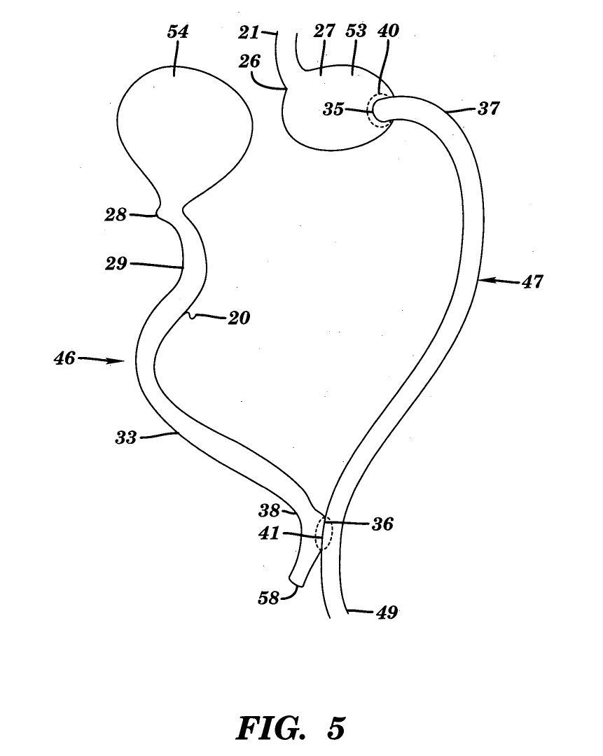

For example, FIG. 5 is a schematic illustration of the end-result of a variation of the foregoing Roux-en-Y gastroplasty, wherein the reduction of the volume of the stomach in which food is lodged while undergoing digestion in the stomach is accomplished by a frank surgical division of the stomach into a divisional stomach pouch 53 and a nonfunctional stomach body 54 that is continuous with afferent jejunal limb of Roux-en-Y gastroplasty 46, rather than by a surgical closing off of the stomach using staples or sutures, as shown in FIG. 3.

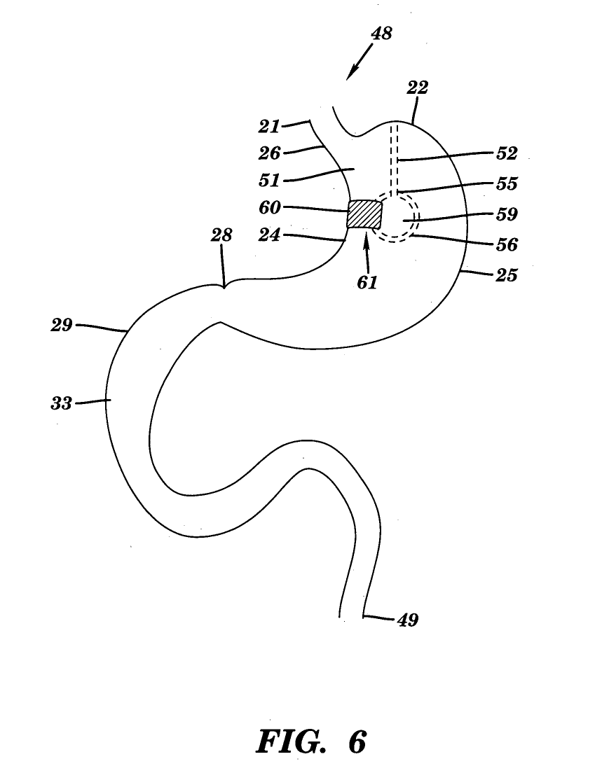

As another example, FIG. 6 is a schematic illustration of the end result of a vertically banded gastroplasty. As shown in FIG. 6, in a vertically-banded gastroplasty (“VBG”) a VBG stomach pouch 51 having a volume of about 15 cc is fashioned as follows. First, the front and back walls of the stomach are stapled together along a vertical line 52 starting at the superior aspect of the fundus 22 of the stomach, and stapling is continued inferiorly for several centimeters. At the inferior terminus 55 of the vertical line of staples 52, a circular stapling instrument is used to continue the stapling together of the front and back walls of the stomach along a circular ring 56. The front and back wall of the stomach apposed by the circular ring of staples 56 is excised leaving a circular window 59. A polypropylene band 60 (Marlex Mesh) is then threaded through circular window 59 and cinched around the lesser curvature of the stomach 24, to form the base of VBG stomach pouch 51, and to fix the size of the outlet 61 of the VBG pouch to the rest of the stomach.

A variation of the VBG procedure threads a silastic ring, rather than a polypropylene band, through circular window 59 and cinches the ring around the lesser curvature of the stomach 24, to form the base of VBG stomach pouch 51, and to fix the size of the outlet 61 of the VBG pouch to the rest of the stomach.

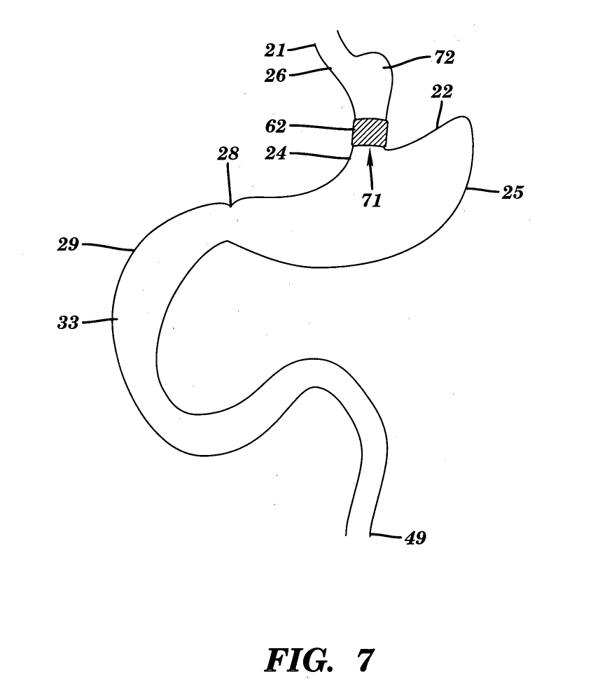

Gastric banding is yet another way to limit food intake. FIG. 7 is a schematic illustration of the end result of a gastric banding procedure, showing an externally applied constricting ring 62 placed completely around the fundus 22 of the stomach at a location just below the gastroesophageal junction 26, thereby creating an hourglass effect, and forming a banded pouch 72, which empties into the rest of the stomach through banded constriction 71.

Other surgical steps and end-results of other surgical modifications of the gastrointestinal tract listed hereinabove are well known in the surgical arts.

A general method is next described for a laboratory investigation of obesity and the reduction of obesity using the foregoing model of an animal having a presurgical substantially normal gastrointestinal tract, which gastrointestinal tract is surgically modified such that postsurgically there is:

[i] a reduction of the volume of the stomach of the animal's gastrointestinal tract; and,

[ii] a reduction in the digestive area of the animal's gastrointestinal tract; and,

[iii] a reduction in the gastric output of the peptide ghrelin; and,

[iv] a reduction in the co-mingling of food with gastric, biliary and pancreatic juices; and,

[v] a permanent reduction in the animal's presurgical weight.

The general method begins with the selection of a plurality of animals having substantially comparable ages and substantially comparable preoperative body weights for exposure to a common controlled laboratory environment, such as, for example, a common cage having, for example, an ambient temperature of about 26° C. and a relative humidity of about 45% and a 12-hour light/dark cycle.

Animals suitable for use in the general method include, for example, murine, ovine, porcine, caprine, canine, feline, and primate animals. Such murine, ovine, porcine, caprine, canine, feline, and primate animals may be transgenic, cloned, or genetically engineered to endow them with certain phenotypes; or, they may be naturally occurring or bred for laboratory use.

The animals are initially permitted free access to a common standardized source of food and water during a period of acclimatization.

The animals are then divided into at least three groups, wherein each member of a first group of the plurality of animals undergoes a sham operation and is thereafter permitted to consume amounts of liquid and solid nutrients ad libitum; and, wherein each member of a second group of the plurality of animals undergoes a surgical modification of its gastrointestinal tract and is thereafter permitted to consume amounts of liquid and solid nutrients ad libitum; and, wherein each member of a third group of the plurality of animals undergoes the sham operation and is thereafter permitted to consume only the mean of the amounts of solid nutrients and liquid nutrients consumed by the members of the second group of the plurality of animals.

Preoperatively, the number of calories consumed per meal, the number of grams of nutrients consumed per meal, and the number of meals taken by each animal is daily or semi-daily measured and recorded.

Preoperatively, the body weight of each animal is daily or semi-daily measured and recorded.

Preoperatively, the total daily or semi-daily caloric intake and the total daily or semi-daily number of grams of nutrients consumed by each animal is daily or semi-daily calculated and recorded.

A surgical modification of the gastrointestinal tract of each of the members of the second group of animals is performed. The surgical modification used in this general method may be selected from the group comprising bariatric surgeries, gastric banding, lap-band adjustable gastric banding, gastric reduction, gastric by-pass, gastrectomy, gastroplasty, Roux-en-Y gastroplasty, vertical banded gastroplasty, silastic ringed vertical gastroplasty, intestinal bypass, restriction operations, weight-loss surgery.

A sham operation on each of the members of the first and third groups of animals is performed. The sham operation may comprise incising and closing the abdominal wall of the members of the first and third groups of animals.

Postoperatively, the number of calories consumed per meal, the number of grams of nutrients consumed per meal, and the number of meals taken by each animal is daily or semi-daily measured and recorded.

Postoperatively, the body weight of each member of each animal is daily or semi-daily measured and recorded.

Postoperatively, the total daily or semi-daily caloric intake and the total daily or semi-daily number of grams of nutrients consumed by each animal is daily or semi-daily calculated and recorded.

Postoperatively, the number of calories consumed per meal, the number of grams of nutrients consumed per meal, and the number of meals taken by each animal is measured and recorded.

All of the animals are sacrificed on a common or non-common postoperative day.

Postmortem, the total daily or semi-daily caloric intake, total daily or semi-daily number of grams of nutrients consumed, number of calories consumed per meal, number of grams of nutrients consumed per meal, number of meals taken, and body weight for each animal are compared.

Postmortem, biological factors relating to biological mechanisms of obesity and reduction of obesity taken from physiological fluids and tissues of each animal are measured, compared, and recorded.

A non-limiting, exemplary specific method is next described for a laboratory investigation of obesity and the reduction of obesity using a Zucker rat having undergone a Roux-en-Y gastroplasty as an exemplary model of an animal having a presurgical weight, and a presurgical substantially normal gastrointestinal tract, which gastrointestinal tract is surgically modified such that postsurgically there is:

[i] a reduction of the volume of the stomach of the animal's gastrointestinal tract; and,

[ii] a reduction in the digestive area of the animal's gastrointestinal tract; and,

[iii] a reduction in the gastric output of the peptide ghrelin; and,

[iv] a reduction in the co-mingling of food with gastric, biliary and pancreatic juices; and,

[v] a permanent reduction in the animal's presurgical weight.

In this non-limiting, exemplary specific method, obese male Zucker rats weighing between about 380 grams and about 420 grams, and aged about 10 to 11 weeks were housed in holding wire cages for one week after their delivery to acclimatize them to the study surroundings, comprising a 12-hour light/dark cycle (light on 05:00-17:00), a room temperature of about 26° C., and a relative humidity of about 45%. The Zucker rats were allowed free access to coarsely ground standard rat chow (Diet No. 5008; Ralston Purina, St. Louis, Mo.) and municipal water.

After acclimatization, the Zucker rats were placed into individual cages, equipped with an Automated Computerized Rat Eatermeter (“ACREM”) developed by the inventor, to measure their food intake, meal size, and number of meals consumed in the course of one week. The ACREM continuously measures meal size, meal number, and food intake without the need preconditioning or pre-training the rats. Access to ground chow occurs via a feeding tunnel that is continuously monitored with photocells. Food consumption was continuously measured via an electronic scale and the size of each meal (“MZ”), the number of meals (“MN”) and the total food intake (“FI) in grams and calories was calculated recorded in real time by a computer.

A meal is defined as a bite or a series of bites preceded and followed by at least 5 minutes of feeding inactivity. Since food intake equals the product of meal size and the number of meals, i.e., FI=MZ×MN, daily or semi-daily food intake can be varied by changing the number of meals, or the size of a meal, or both. Thus, it is important to measure each component of FI, because MZ and MN are regulated independently by different portions of the central nervous system.

Following the week of metered feeding, the Zucker rats were randomly divided into three groups:

[i] a Control Group that was to be fed ad libitum, following a sham operation; and,

[ii] a Gastric Bypass (“GB”) Group that was to be fed-ad libitum, following a Roux-en-Y gastroplasty; and,

[iii] a Pair Fed (“PF”) Group that was to be fed the mean of the amounts of the liquid nutrients and solid nutrients consumed by the GB group, following a sham operation, i.e., without having undergone the Roux-en-Y gastroplasty, the PF group was fed only the mean amount of liquid nutrients and solid nutrients consumed by the GB group, which had undergone the Roux-en-Y gastroplasty.

After 18 hours of food deprivation, all of the GB Zucker rats underwent reconstructive gastrointestinal surgery with the Roux-en-Y gastroplasty, as described hereinabove. All of the Control Group Zucker rats and all of the Pair Fed Group Zucker rats underwent a sham operation in which their abdomens were simply incised and then closed.

Postoperatively, and following a period of recovery, all Zucker rats were returned to their ACREM cages. Depending on the hydration status of the Zucker rats, normal saline (30 ml) was injected into the abdominal wall for the first few postoperative days. Postoperatively pain medication was not required. All of the Zucker rats were allowed resume eating and drinking 24 hours after surgery.

A liquid diet (Boost, Mead-Johnson, Evansville, Ind.; 1 kcal/g) was provided for the first 4 days. Thereafter, for 6 days, coarsely ground Purina chow (Diet No. 5008, 3.5 kcal/g) was added to their diets. Food was provided ad libitum to the GB group and the Control group, but the PF Group was given only the mean of the amounts of the liquid and solids consumed by the GB group.

All rats were allowed water ad libitum. FI, MZ, MN, and body weight (“BW”) were measured or semi-daily at approximately 09:00 and 21:00.

On Day 10 postoperatively, all of the Zucker rats were sacrificed under anesthesia. Following their sacrifice, mixed venous and arterial blood was obtained to measure glucose, free fatty acid (FFA), triglyceride (TG), and insulin concentrations, using standard commercial radioimmunoassay (“RIA”) kits.

The size of the surgically fashioned gastric pouch was measured and its volume was calculated. The diameter of gastrojejunostomy anastomosis was also measured. Liver fat content was assayed, and retroperitoneal and epididymal fat pads were weighed.

The caloric intake of the Control Group, GB Group and PF Group were compared using the two-sample Student t test and the nonparametric Mann-Whitney U test. The differences in body weights between Zucker rats of the Control Group and the Zucker rats of the GB group were examined by Student's t test. A P value <0.05 was regarded as statistically significant.

As shown in Table 1 below, preoperatively, there was no significant difference in caloric intake, MZ, or MN among groups. Postoperatively, compared with the Control Group, caloric intake was significantly decreased after Roux-en-Y gastroplasty in the GB Group (P<0.05). The decrease in caloric intake correlated with a measured decrease in MZ, which was significantly reduced in the GB Group as compared with the Control Group. Moreover, the MN of the GB Group was significantly decreased during the entire post-operative period as compared with the Control Group.

TABLE 1
Calorie Intake, Meal Size, and Meal Number Changes before and after
Gastric Bypass with Roux-en-Y Operation

| | Control | | GB | |
|---|---|---|---|---|
| | Postoperative Day | | | |
| | Preop | 5-10 | Preop | 5-10 |
| Calorie intake | 95.5 ± 1.9 | 102.1 ± 2.1 | 94.9 ± 2.4 | 31.2 ± 2.7* |
| Meal size | 2.8 ± 0.1 | 3.5 ± 0.2 | 2.7 ± 0.13 | 1.4 ± 0.2 |
| Meal number | 10.4 ± 0.4 | 8.7 ± 0.3 | 10.5 ± 0.4 | 7.2 ± 0.3* |

*P < 0.05.

As shown in Table 2 below, postoperatively, serum glucose concentration was significantly decreased (P<0.05) in the GB Group as compared to the Control Group. Serum insulin concentration was also significantly decreased in both the GB Group and PF Group as compared to the Control Group

Also shown in Table 2, the free fatty acid (“FFA”) concentration was significantly decreased (P<0.05) in the GB Group as compared to the Control Group, while the triglyceride (“TG”) concentration was significantly lower in the PF group as compared to the Control Group. The TG concentration was also lower, but not significantly so in the GB Group as compared to the Control Group.

Further shown in Table 2, retroperitoneal and epididymal fat weight was significantly decreased (P<0.05;) in the GB Group as compared to the Control Group.

TABLE 2
Effect of Gastric Bypass with Roux-en-Y Operation on Biochemical
Parameters in Zucker Rats

| | Control | PF | GB |
|---|---|---|---|
| Glucose (mg/dl) | 196.0 ± 10.7 | 150.5 ± 6.4 | 164.7 ± 9.8* |
| Insulin (ng/ml) | 7.1 ± 0.6 | 3.0 ± 0.4* | 4.9 ± 0.7* |
| Free fatty acids | 0.58 ± 0.03 | 0.68 ± 0.04 | 0.37 ± 0.03* |
| (mmol/L) | | | |
| Triglyceride (mg/dl) | 356.4 ± 32.0 | 157.5 ± 14.4* | 267.9 ± 29.1 |
| Retroperitoneal fat (g) | 22.9 ± 0.7 | 18.3 ± 0.5* | 17.2 ± 0.7* |
| Epididymal fat (g) | 15.4 ± 0.5 | 12.1 ± 0.5* | 12.3 ± 0.5* |
| Liver lipid content | 66.9 ± 5.4 | 45.0 ± 3.5 | 54.0 ± 4.0 |
| (mg/g) | | | |

*P < 0.05.

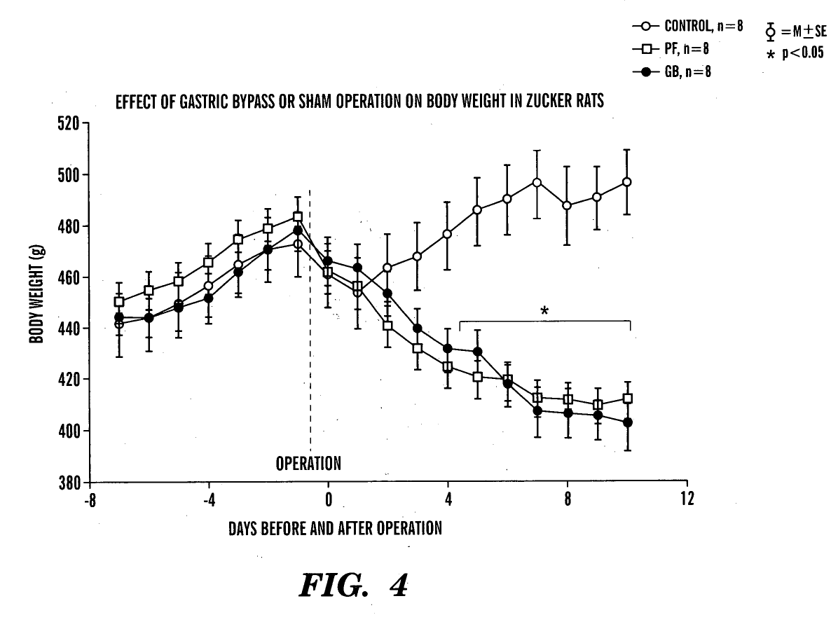

As shown in the graph appearing in FIG. 4, preoperatively, there was no substantial difference in body weight gain among the groups. Postoperatively, in all groups there was a decrease in body weight, over 2 to 4 days, attributed to the effects of surgery and anesthesia. Thereafter, body weight in the Control Group gradually rose by 5.2 g/day and reached 497.1±12.4 g by Day 10, when the Zucker rats were sacrificed. In both the GB Group and the PF Group, body weight decreased continuously until these rats were sacrificed. The mean weight loss was 75.5 g during the 10 days after the operation. Terminal body weight was significantly lower (P<0.05) in the GB Group than in the Control Group.

In this exemplary non-limiting, specific method using the exemplary animal model, a decrease in food intake concomitant with a decrease in body weight and body fat in Zucker rats having had a Roux-en-Y gastroplasty was demonstrated relative to controls.

The decrease in food intake resulted from an observed decrease in meal size, owing to the surgical reduction in gastric volume. This decrease in meal size was not accompanied by an expected compensatory rise in the number of meals in order to maintain the level of preoperative food intake. Rather, in Zucker rats that underwent Roux-en-Y gastroplasty, the postoperative decrease in meal size was accompanied by a reduction in the meal number.

The dissociation in the relationship between meal size and meal number is characteristically seen in a variety of disease states that cause anorexia, and reflects a change in the neurotransmitter relationship between dopamine and serotonin in the hypothalamus. Thus, a similar change in the neurotransmitter relationship between dopamine and serotonin in the hypothalamus may be postulated to occur following Roux-en-Y gastroplasty, a hypothesis which can be tested with this exemplary non-limiting, specific method using the exemplary animal model.

In this exemplary non-limiting, specific method using the exemplary animal model, the messenger RNA (“mRNA”) coding for the synthesis of the protein ghrelin in the stomach was also measured. Ghrelin is a peptide produced primarily by the oxytincic cells of the gastric fundus, and it is the primary appetite stimulatory peptide acting on the orexigenic neuropeptide Y in the hypothalamus. It was noted that ghrelin mRNA expression in the stomach decreased, as did the concentration of serum ghrelin in Zucker rats having undergone the Roux-en-Y gastroplasty. This decreased the stimulatory signal sent to the brain to eat. Significantly, a significant increase in serum ghrelin concentration occurred in the PF Group, which would have stimulating the PF Zucker rat to eat more food, had it been made available.

I claim:

1. A surgically modified animal comprising an animal having a preoperative weight, a preoperative endogenous ghrelin production and a preoperative substantially normal animal gastrointestinal system that has been surgically modified, wherein said surgical modification reduces the volume of the stomach of said gastrointestinal tract and reduces the digestive area of said gastrointestinal tract; and, wherein postoperatively, said surgically modified animal exhibits a substantially permanent reduction of said preoperative weight and a substantially permanent reduction in said preoperative endogenous ghrelin production.

2. The animal model of claim 1, wherein said animal is selected from the group comprising murine, ovine, porcine, caprine, canine, feline, and primate animals.

3. The animal model of claim 1, wherein said animal is selected from the group comprising transgenic murine, ovine, porcine, caprine, canine, feline, and primate animals.

4. The animal model of claim 1 wherein said animal is a Zucker rat.

5. The animal model of claim of claim 1, wherein said animal is selected from the group comprising genetically modified murine, ovine, porcine, caprine, canine, feline, and primate animals.

6. The animal model of claim 1, wherein said animal is selected from the group comprising cloned murine, ovine, porcine, caprine, canine, feline, and primate animals.

7. The animal model of claim 1, wherein said surgical modification is selected from the group comprising bariatric surgeries, biliopancreatic diversion, gastric banding, gastric reduction, gastric by-pass, gastrectomy, gastrocolostomy, gastroduodenostomy, gastroenterocolostomy, gastroenteroplasty, gastroenterostomy, gastroenterotomy, gastroesophagostomy, gastrogastrostomy, gastroileostorny, gastrojejunostomy, gastroplasty, vertical banded gastroplasty, intestinal bypass, restriction operations, and weight-loss surgery.

8. The animal model of claim 4 wherein said Zucker rat has undergone a Roux-en-Y gastroplasty.

9. A method for performing a Roux-en-Y gastroplasty on a Zucker Rat comprising the steps of:
a. administering anesthesia;
b. shaving and sterilizing the abdomen of said Zucker rat;
c. incising the abdomen of said Zucker rat;
d. identifying the terminal esophagus, lesser curvature of the stomach, and greater curvature of the stomach of said Zucker rat;
e. dissecting the terminal esophagus, lesser curvature of the stomach, and greater curvature of the stomach of said Zucker rat free of their surrounding supportive and membranous tissues;
f. creating a gastric pouch having a volume of about 20% of the presurgical stomach of said Zucker rat;
g. dividing the jejunum of said Zucker rat at a location about 16 cm below the ligament of Treitz, thereby creating a distal portion of the divided jejunum and a proximal portion of the divided jejunum of said Zucker rat;
h. performing a gastrojejunostomy on said Zucker rat by anastomosing the distal portion of the divided jejunum of said Zucker rat to a site on the anterior surface of the gastric fundus of said Zucker rat;
i. performing a jejunojejunostomy on said Zucker rat by anastomosing the proximal aspect of the divided jejenum of said Zucker rat to a site at a distance of about 10 cm below the site of the gastrojejunostomy;
j. closing the stump of the proximal portion of the divided jejunum of said Zucker rat;
k. closing the incised abdomen of said Zucker rat.

10. The method of claim 9, wherein said step of creating a gastric pouch further comprises the steps of:
a. placing a first row of surgical staples across the stomach of said Zucker rat about 2-3 mm below the gastroesophageal junction of said Zucker rat;
b. placing a second row of surgical staples across the stomach of said Zucker rat about 4-5 mm below the gastroesophageal junction of said Zucker rat;
c. reinforcing said first and second row of surgical staples with sutures.

11. A method for investigating the biological mechanisms of obesity and reducing obesity comprising the steps of:
a. selecting a plurality of animals for confinement in a common controlled laboratory environment;
b. dividing said plurality of animals into at least three groups, wherein each member of a first group of said plurality of animals undergoes a sham operation and is thereafter permitted to consume amounts of liquid nutrients and solid nutrients ad libitum; and, wherein each member of a second group of said plurality of animals undergoes a surgical modification of its gastrointestinal tract and is thereafter permitted to consume amounts of liquid nutrients and solid nutrients ad libitum; and, wherein each member of a third group of said plurality of animals undergoes said sham operation and is thereafter permitted to feed only a mean of said amounts of liquid nutrients and solid nutrients consumed by said members of said second group of said plurality of animals;
c. daily measuring and recording a preoperative number of calories consumed per meal, a preoperative number of grams of nutrients consumed per meal, and a preoperative number of meals taken by each member of each of said first, second and third groups of said plurality of animals;
d. daily measuring and recording a preoperative body weight of each member of each of said first, second and third groups of said plurality of animals;
e. daily calculating and recording a preoperative total daily caloric intake and a preoperative total daily number of grams of nutrients consumed by each member of each of said first, second and third groups of said plurality of animals;
f. performing said surgical modification of said gastrointestinal tract of each of said members of said second group of said plurality of animals;
g. permitting each of said members of said second group of said plurality of animals to resume eating and drinking about 24 hours postoperatively;
h. performing said sham operation on each of said members of said first and second groups of said plurality of animals;
i. permitting each of said members of said first and second groups of said plurality of animals to resume eating and drinking about 24 hours postoperatively;
j. feeding each member of said first and second groups of said plurality of animals a diet of liquid nutrients ad libitum for about 4 postoperative days.
k. additionally feeding each member of said first and second groups of said plurality of animals solid nutrients ad libitum on about a 5th postoperative day and continuing until a sacrifice of said members of said first and second groups.
l. feeding each member of said third group of said plurality of animals a diet of liquid nutrients in an amount equal to a mean amount of liquid nutrients consumed by said members of said second group for said about 4 postoperative days.
m. additionally feeding said members of said third group of said plurality of animals an amount equal to a mean amount of solid nutrients consumed by said members of said second group of said plurality of animals, beginning on about a 5th postoperative day and continuing until a sacrifice of said members of said third group.
n. daily measuring and recording a postoperative number of calories consumed per meal, a postoperative number of grams of nutrients consumed per meal, and a postoperative number of meals taken by each member of each of said first, second and third groups of said plurality of animals;
o. daily measuring and recording a postoperative body weight of each member of each of said first, second and third groups of said plurality of animals;
p. daily calculating and recording a postoperative total daily caloric intake and a postoperative total daily number of grams of nutrients consumed by each member of each of said first, second and third groups of said plurality of animals;
q. daily calculating and recording a postoperative number of calories consumed per meal, a postoperative number grams of nutrients consumed per meal and a postoperative number of meals taken by each member of each of said first, second and third groups of said plurality of animals;
r. sacrificing said plurality of animals on a common postoperative day;
s. postmortem, comparing said preoperative and said postoperative total daily caloric intake, said preoperative and said postoperative total daily number of grams of nutrients consumed, said preoperative and said postoperative number of calories consumed per meal, said preoperative and said postoperative number of grams of nutrients consumed per meal, said preoperative and said postoperative number of meals taken, and said preoperative and said postoperative body weight for each member of said first, second and third groups of said plurality of animals;
t. postmortem, measuring and comparing biological factors relating to biological mechanisms of obesity and reduction of obesity.

12. The method of claim 11, wherein said step of selecting a plurality of animals for confinement in a common controlled laboratory environment further comprises selecting a plurality of animals having substantially comparable ages and preoperative body weights.

13. The method of claim 11, wherein said step of selecting a plurality of animals for confinement in a common controlled laboratory environment further comprises selecting said plurality of animals from the group comprising murine, ovine, porcine, caprine, canine, feline, and primate animals.

14. The method of claim 11, wherein said step of selecting a plurality of animals for confinement in a common controlled laboratory environment further comprises selecting said plurality of animals from the group comprising transgenic murine, ovine, porcine, caprine, canine, feline, and primate animals.

15. The method of claim 11, wherein said step of selecting a plurality of animals for confinement in a common controlled laboratory environment further comprises selecting said plurality of animals from the group comprising genetically modified murine, ovine, porcine, caprine, canine, feline, and primate animals.

16. The method of claim 11, wherein said step of selecting a plurality of animals for confinement in a common controlled laboratory environment further comprises selecting said plurality of animals from Zucker rats.

17. The method of claim 11, wherein said step of selecting a plurality of animals for confinement in a common controlled laboratory environment further comprises selecting said plurality of animals from the group comprising cloned murine, ovine;, porcine, caprine, canine, feline, and primate animals.

18. The method of claim 11, wherein said step of performing said surgical modification of said gastrointestinal tract of each of said members of said second group of said plurality of animals further comprises selecting said surgical modification from the group comprising bariatric surgeries, biliopancreatic diversion, gastric banding, gastric reduction, gastric by-pass, gastrectomy, gastrocolostomy, gastroduodenostomy, gastroenterocolostomy, gastroenteroplasty, gastroenterostomy, gastroenterotomy, gastroesophagostomy, gastrogastrostomy, gastroileostomy, gastrojejunostomy, gastroplasty, vertical banded gastroplasty, intestinal bypass, restriction operations, and weight-loss surgery.

19. The method of claim 11, wherein said step of dividing said plurality of animals into at least three groups further comprises selecting said second group of animals from Zucker rats and surgically modifying said gastrointestinal tract of each of said members of said second group by means of a Roux-en-Y gastroplasty.

20. The method of claim 11, wherein said sham operation comprises opening and closing the abdomen of said members of said first and third groups of said plurality of animals.

21. The method of claim 11, wherein said step of selecting a plurality of animals for confinement in a common controlled laboratory environment further comprises confining said plurality of animals to a common cage having a common standardized source of food and water for a period of about one week after their selection to acclimatize them to their surroundings.

22. The method of claim 11, wherein said step of selecting a plurality of animals for confinement in a common controlled laboratory environment further comprises providing said controlled environment with an ambient temperature of about 26° C., a relative humidity of about 45%, and a 12-hour light/dark cycle.

23. The method of claim 11, wherein said step dividing said plurality of animals into at least three groups further comprises, initially confining each member of said plurality of animals to an individual cage for a period of about 1 week, equipped with a device to continuously feed, measure, calculate and record said preoperative total daily number of grams of nutrients consumed, said preoperative number of calories consumed per meal, said preoperative number of grams of nutrients consumed per meal, said preoperative number of meals taken, and said preoperative body weight for each member of said first, second and third groups of said plurality of animals.
